SEQUENCE LISTING (2 sequences)

SEQ ID NO: 1 - DNA, 561 bp, Soybean
cggggtaccttggtactggtgctactgaccagcaaggcaaactcagcggaaactgtttctttcagctggaacaagttcgtgccgaagcaaccaaacatgatcctccaaggagacgctattgtgacctcctcgggaaagttacaactcaataaggttgacgaaaacggcaccccaaaaccctcgtctcttggtcgcgccctctactccacccccatccacatttgggacaaagaaaccggtagcgttgccagcttcgccgcttccttcaacttcaccttctatgcccctgacacaaaaaggcttgcagatgggcttgccttctttctcgcaccaattgacactaagccacaggttatcttggtcttttcaacgaaaacgcagtctggtgatcaagtcgtcgctgttgagtttgacactttccggaactcttgggatccaccaaatccacacatcggaattaacgtcaattctatcagatccatcaaaacgacgtcttgggatttggccaacaataaagtagccaaggttctcattacctatgatgcctccaccggtaccccg

SEQ ID NO: 2 - DNA, 1187 bp, Homo sapiens
atgcctttggattagggcaaactctaggatatttaagtgaattccctgaggaatgtggactctgtaagcaggtgagtgcattattctctgcttctctctctccacagggccgtcgttcaccctcctccaccttgtcccctgcactgggaggcaaccacaacaggcacggcccatgctcctgcaccacctggcttctgcttgggtgtggatgataacaggcacctgcaggagatgggagcatgcggggagaagtaactcagggttttcatttccctcactccctctggacagctctgtggttccgtaatcattgccgtcctctacctacagccacaggcatgtgggtctgcccctagtgaaagctacagatttccttgggttctggaaactgctcccttcgttgctctttcaagcttcaagatgaaaacagtttcctgccaggaataatcccagggagcttcagcgccctttgtggctttcttagccctgccggcacctgtgtagaaggtgccatctcaggccagcgcggtggctcaaacgtgtaatctcagcaccttgagaggctgaggccagaggatcacctgaggttgggagttcaagaccagcctgaccaacatggagaaaccctgtctctactaaaaataaaaaattagctgggcgtggtggcgcatgcctgtaatcccagctactcgggaggctgaggcaggagaattgcttgaacccaggaggcagaggttgtggtgagccgagatcacaccattgcactccagcctgggcaacaagagtgaaactcagtctcggaaaaaaaaaaaaggtgccatctctttcctgccaggtccctgactgaccacagggtgctcccacaaaaggagaagtgacaagaatgtatttaagacattgcactaacacatctattcatgatgttaattcaaaaaattgacttactacaataaaagggaaaaataagagtattctggaaacagagcaggaaggaaggcaaaggtgaaaacaatcaatctggggcatctgagaagccccaagtgcagaggctgccctgagtctttagaggacaagaaacagaacacacgacccaaaagtgagagacagagcctggccggagcaggatgataacggctctcctacagagtactattcctgtaaatctctgacgagaggggtgagatcaa

(19) United States
(12) Patent Application Publication
(10) Pub. No.: US 2016/0130633 A1
(43) Pub. Date: May 12, 2016

(54) METHOD AND APPARATUS FOR AMPLIFYING DNA FRAGMENT BASED ON CONTROLLING PH CHANGE OF REACTION SOLUTION

(71) Applicant: SHANGHAI INTISTUTE OF APPLIED PHYSICS, CHINESE ACADEMY OF SCIENCE, Shanghai (CN)

(72) Inventors: Yi ZHANG, Shanghai (CN); Gang LIU, Shanghai (CN); Qing HUANG, Shanghai (CN); Chunhai FAN, Shanghai (CN)

(21) Appl. No.: 14/897,204

(22) PCT Filed: Jun. 9, 2014
(86) PCT No.: PCT/CN2014/079482

§ 371 Date: Dec. 9, 2015

(30) Foreign Application Priority Data
Jun. 9, 2013 (CN) 201310231925.2

(51) Int. Cl. C12Q 1/68; C12M 1/34; C12M 1/40

(52) U.S. Cl. CPC C12Q 1/686; C12M 21/18; C12M 41/26

(57) ABSTRACT

The present invention provides a method and apparatus based on a DNA fragment amplified by changing the pH value of a control reaction solution. Specifically, the present invention provides a method for nucleic acid amplification, comprising the following steps: (a) under conditions of pH 10-14 alkalinity, melting of a double-stranded nucleic acid molecule; (b) under conditions of pH 5-8 neutrality and near-neutrality, annealing of the melted nucleic acid molecule and a primer; and in the presence of a nucleic acid polymerase, causing the primer bound to the single-stranded nucleic acid molecule to extend to form an amplified double-stranded nucleic acid molecule.

### FIELD OF THE INVENTION

The present invention relates to the field of nucleic acid detection. In particular, the present invention relates to a nucleic acid amplification method based on controlling double-stranded nucleic acid denaturation—annealing process by changing pH value of the solution.

### BACKGROUND OF THE INVENTION

PCR technology is widely used in areas such as life science experiments and biomolecular testing, and it plays an incalculable role in development of life sciences. Because it is simple to prepare a nucleic acid sample to be amplified, only small amount of sample is required, it is simple to operate and it is fully automatic in amplification process, it is gradually applied to the relevant fields. In recent years, PCR technology has very exciting applications in many fields such as pathology testing, early warning of disease, epidemic virus isolation, archeology, forensic science, etc.

Since PCR technology is highly irreplaceable, the PCR technology has been in continuous development and innovation since its birth. Current PCR technique starts from the changes of temperature, by controlling the denaturing, annealing and extension process of DNA molecule carried out at different temperatures and applying various bases, primers, inorganic ions, buffers and DNA polymerization enzymes necessary for DNA replication, so as to complete amplification of DNA molecules with high fidelity. However, this method is obviously limited. First, each PCR reaction cycle involves substantial temperature change. Since the temperature change is a slow process, the entire PCR reaction requires at least two hours. Second, in each cycle, the reaction temperature reciprocally changes within the range of 94-36-72° C., thus leading to high energy consumption. Therefore, the use cost of the present PCR is relatively high. Another side effect of high electric energy consumption is the damage to environment. Third, in addition to real-time quantitative PCR, conventional PCR reactions can not monitor reaction procedure, and it can not timely intervene DNA amplification effect. These deficiencies restrict PCR technology from further development and become obstacle for wide use of PCR.

Therefore, a PCR technology having low-energy consumption, having short reaction time and capable of being timely intervened is needed in the field.

### DETAIL OF THE INVENTION

The purpose of the present invention is to provide a double-stranded nucleic acid denaturation—annealing process controlled by adjusting pH value of system.

Another object of the present invention is to provide a simple, efficient PCR technique which can be conducted at normal temperature.

In the first aspect of the present invention, it provides a method of nucleic acid amplification, which comprises:

(1a) a denaturing step: in an alkaline condition of pH 10-14, unwinding a double-stranded nucleic acid molecule in an amplification system;

(1b) an annealing and extension step: at a neutral or near-neutral condition of pH 5-8, annealing single-stranded nucleic acid molecules and primers in the amplification system, and in presence of a nucleic acid polymerase, extending the primers binding to the single-stranded nucleic acid molecules, thereby forming amplified double-stranded nucleic acid molecules.

In another preferred embodiment, the method comprises repeating the above steps (a) and (b) for at least 15-60 times, and preferably 20-45 times.

In another preferred embodiment, in the steps (a) and/or (b), reaction temperature is 10-70° C.; and preferably, reaction temperature is 15-45° C.

In another preferred embodiment, during the entire amplification process, the temperature of the amplification system maintains at 10-70° C., and preferably at 15-45° C.

In another preferred embodiment, the pH in step (a) is preferably 11.5-12.5.

In another preferred embodiment, the pH in step (b) is preferably 6.5-7.5.

In each steps, the preferred time range is as follows: step (a) >20 seconds, and step (b) >60 seconds.

In step (a) it is preferably 20-120s, more preferably 25-80s, and most preferably 35-60s; and in step (b) it is preferably 60-300s, more preferably 60-150s, most preferably 100-150s.

In another preferred embodiment, the nucleic acid molecules comprise DNA, RNA, or DNA-RNA hybrid molecules.

In another preferred embodiment, the amplification system comprises DNA to be amplified, dNTPs, primers, polymerase and magnesium ion.

In another preferred embodiment, in steps (a) and/or step (b), the pH value of the amplification system is adjusted by adding alkaline solution or acid solution.

In another preferred embodiment, the pH value of the amplification system is adjusted to 10-14 by adding alkaline solution into the amplification system. In another preferred embodiment, the alkaline solution comprises strong alkaline solutions such as NaOH, KOH, Ca(OH)₂ solutions, etc., and BR buffer (pH=11-14), disodium hydrogen phosphate—sodium hydroxide buffer (pH=10-12) and any other buffer solution which can adjust the pH value of the reaction system to alkaline (pH>10).

In another preferred embodiment, the pH value of the amplification system is adjusted to 5-8 by adding acid solution into the amplification system.

In another preferred embodiment, the acid solution comprises strong acid solutions such as HCl solution, H₂SO₄ solution, HNO₃ solution, H₃PO₄ solution, and BR buffer (pH=0-3), phosphate buffer, Tris-HCl buffer, HEPES buffers and any other buffer solution which can adjust the pH value of the reaction system to neutral.

In another preferred embodiment, the method further comprises: adjusting the pH value of the amplification system by electrochemical methods.

In another preferred embodiment, in the step (a) and/or step (b), it further comprises determining electric potential of the amplification system by electrochemical methods so as to determine the pH value of the amplification system.

In another preferred embodiment, in the step (a) and/or step (b), it further comprises adjusting electric potential of the amplification system by electrochemical methods so as to adjust the pH value of the amplification system.

In another preferred embodiment, the method of amplifying the nucleic acid is polymerase chain reaction (PCR).

In another preferred embodiment, the nucleic acid polymerase is an enzyme having DNA polymerase activity. Preferably, the nucleic acid polymerase comprises: E. coli DNA polymerase I, Klenow fragment (DNA polymerase I large fragment), E. coil DNA polymerase II, E. coli DNA polymerase III, T4 DNA polymerase, T7 DNA polymerase, DNA polymerase a, DNA polymerase β, DNA polymerase y, DNA polymerase 8, Taq DNA polymerase, Tth DNA polymerase, pfu DNA polymerase, Vent DNA polymerase, Bca Best DNA polymerase, Sac DNA polymerase, Iproof DNA polymerase, KOD DNA polymerase, Phusion DNA polymerase, U11traPF™ DNA polymerase, LA Tag DNA polymerase, or Super Tag DNA polymerase.

In another preferred embodiment, the nucleic acid polymerase can tolerate pH 5-14.

In another preferred embodiment, the nucleic acid polymerase can tolerate pH 6-9.

In another preferred embodiment, the step (b) further comprises a step of refilling nucleic acid polymerase.

In another preferred embodiment, the addition quantity of nucleic acid polymerase is 10U/cycle period.

In the second aspect of the present invention, it provides an equipment for amplifying a nucleic acid, wherein the equipment comprises:

(3i) an device to place a vessel in which a nucleic acid amplification reaction is conducted, wherein the vessel is used to accommodate an amplification system for conducting a nucleic acid amplification reaction;

(3ii) an alkaline solution adding device which is used to add alkaline solution into the amplification system so as to adjust pH of the amplification system to alkaline condition;

(3iii) an acid solution adding device which is used to add acid solution into the amplification system so as to adjust pH of the amplification system to acidic or neutral condition;

(3iv) a pH determining device which is used for accurately controlling pH value of the system.

In another preferred embodiment, one or more devices in (i)-(iv) are included in an electrochemical working station.

In another preferred embodiment, the device to place the vessel in which the nucleic acid amplification reaction is conducted is a DNA reaction chip.

In another preferred embodiment, the pH determining device comprises an indicator, an acidometer, or a pH potentiometer.

In another preferred embodiment, the equipment further comprises a nucleic acid polymerase adding device.

In the third aspect of the present invention, it provides an equipment for amplifying a nucleic acid, wherein the equipment comprises:

(4i) an amplification device to place a vessel in which a nucleic acid amplification reaction is conducted, wherein the vessel is used to accommodate an amplification system for conducting a nucleic acid amplification reaction; and

(4ii) an electrochemical working station which is used to regulate and detect pH of the reaction system.

In another preferred embodiment, the amplification device comprises a micro fluidic chip, a DNA reaction chip, an eppendorf tube, or a culture flask.

In another preferred embodiment, the electrochemical working station connects to the amplification device by wire.

In another preferred embodiment, the electrochemical working station adjusts pH of the amplification system to a desired value by applying a predetermined voltage to the system.

In another preferred embodiment, the equipment further comprises an adding device which is used to add nucleic acid polymerase.

In another preferred embodiment, the amplification device is a micro fluidic chip.

In another preferred embodiment, the equipment adjusts pH value of the amplification system by electrochemical methods.

In another preferred embodiment, the device produces an electric current by changing a potential difference between a working electrode and a reference electrode on the chip and causes a corresponding degree of decomposition of electrolytes in DNA reaction solution, thereby causing a pH change.

In another preferred embodiment, the micro fluidic chip comprises a working electrode and/or a reference electrode.

In another preferred embodiment, the working electrode is Ag/AgCl electrode.

In another preferred embodiment, the reference electrode is IrO₂ electrode.

In another preferred embodiment, the micro fluidic chip comprises a reaction area containing DNA reaction solution (or R area) and /or a control area containing electrolyte solution (or C area).

In another preferred embodiment, the working electrode connects to the C area.

In another preferred embodiment, the reference electrode connects to the R area.

In another preferred embodiment, the C area and the R area are semi-closed connected.

In the forth aspect of the present invention, it provides a method to amplify nucleic acid, wherein it comprises:

(1) providing an amplification system which comprises a DNA template to be amplified, primers, dNTPs, magnesium ions, and a polymerase; and

(2) adjusting pH value of the amplification system by an electrochemical method so as to achieve denaturing and annealing of nucleic acid molecules, and to conduct amplification of the nucleic acid.

In another preferred embodiment, the method comprises:

In step (1), it comprises providing an nucleic acid amplification equipment in the third aspect of the present invention, and adding the DNA template, the primers, dNTPs, magnesium ions, and the polymerase into the equipment so as to form an amplification system; and

in step (2), the electrochemical method is by using an electrochemical working station.

In another preferred embodiment, the method further comprises: before the step (1), connecting the equipment to place the vessel in which the nucleic acid amplification reaction is conducted in the third aspect of the present invention and the electrochemical working station by wire, and determining a standard potential-pH curve; and setting an electrode potential of the electrochemical working station based on the determined standard potential-pH curve.

In another preferred embodiment, the standard curve is determined before a single amplification.

In another preferred embodiment, the standard curve is not separately determined when the reaction system is continuously used.

In another preferred embodiment, the electrochemical working station adjusts pH value of the system by applying a predetermined voltage to the amplification system.

In another preferred embodiment, the polymerase is DNA polymerase I or Taq enzyme.

In the fifth aspect of the present invention, it provides a method to amplify nucleic acid by using an electrochemical method, wherein it comprises one or more of the following steps:

determining the relationship between electrical parameters and pH of solution in the amplification solution system;

setting the electrical parameters; unwinding double-stranded nucleic acid in the amplification system by controlling pH to an alkaline condition of pH 10-14 through solution electrochemical reaction;

setting the electrical parameters, annealing a single-stranded nucleic acid molecule and a primer in the amplification system, and in the presence of a nucleic acid polymerase, extending the primer binding to the single-stranded nucleic acid molecule to form an amplified double-stranded nucleic acid molecule by controlling pH at a neutral or near-neutral condition of pH 5-8 through solution electrochemical reaction.

In another preferred embodiment, the electrical parameters include potential, current, resistance, capacitance, conductance, or electric quantity.

In another preferred embodiment, the method comprises repeating steps (1), (2) and (3) for at least 15-60 times, and preferably 20-45 times.

In the sixth aspect of the present invention, it provides a method to detect nucleic acid, which comprises:

amplifying a nucleic acid to be detected by using the method in the first, the fourth or the fifth aspect of the present invention; and

detecting the amplified nucleic acid.

In another preferred embodiment, the amplified nucleic acid is detected by an electrophoresis method.

In another preferred embodiment, before the detection, the products is digested with restriction enzyme and sequenced for subsequent detection.

In the seventh aspect of the present invention, it provides a method for denaturing—annealing a nucleic acid, wherein it comprises:

(10a) providing a reaction system containing a nucleic acid;

(10b) adjusting pH of the reaction system to pH=10-14 so as to denature DNA, and adjusting pH of the reaction system to pH=5-8 so as to anneal the DNA;

wherein the denaturing—annealing process can be conducted repeatedly.

In another preferred embodiment, the denaturing—annealing process can be repeated for at least 2 cycles, preferably at least 10 cycles, more preferably at least 20 cycles, and most preferably at least 30 cycles.

In another preferred embodiment, adjusting pH of the system is achieved by an electrochemical method.

It should be understood that, in the present invention, each of the technical features specifically described above and below (such as those in the Examples) can be combined with each other, thereby constituting new or preferred technical solutions which need not be specified again herein.

### DESCRIPTION OF THE DRAWINGS

FIG. 1 shows an electrophoresis comparison between a DNA treated with strong acid and strong alkali and a normal DNA.

FIG. 2 shows an electrophoresis comparison of soybean Lectin DNA sequence after 30 cycles of denaturing, annealing and amplifying under different pH values in Example 2.

FIG. 3 shows an electrophoresis comparison between DNA amplified by acid-alkali method in different annealing pH value in Example 3.

FIG. 4 shows electrophoresis test results of the optimal denaturation and annealing time of soybean Lectin gene in BR buffer in Example 4.

FIG. 5 shows an electrochemical—micro fluidic chip in Example 5.

FIG. 6 shows relationship between control voltages of electrochemical working tation -DNA reaction chip and pH value of solution in Example 6.

FIG. 7 shows amplification in the electrochemical working station -DNA reaction chip in Example 6.

FIG. 8 shows electrophoresis (after digestion) of amplification product of soybean Lectin gene in the electrochemical working station -DNA reaction chip in Example 7.

FIG. 9 shows an amplification reaction in the electrochemical working station-DNA reaction control chip in Example 8, wherein E. coli DNA polymerase I, E. coli DNA polymerase II, E. coli DNA polymerase III, Bca Best DNA polymerase, U11traPF™ DNA polymerase were used as polymerase, respectively.

FIG. 10 shows an electrophoresis graph of comparison between the soybean Lectin DNA sequences amplified by the electrochemical reaction working station-DNA reaction chip in Example 6 and a conventionally amplified PCR product.

FIG. 11 shows an agarose electrophoresis graph of amplification of human genome chromosome 6th gene fragment in an electrochemical working station -DNA reaction chip in Example 8.

### EMBODIMENTS FOR CARRYING OUT THE INVENTION

Through long-term and intensive research, the inventors have unexpectedly discovered that by adjusting pH value of an amplification system, not only a double-stranded nucleic acid can be effectively denatured and annealed, but also the denaturing-annealing process is essentially reversible, and therefore it can be conducted in cycle for multiple times. This feature is particularly suitable for development of a nucleic acid amplification technology which is based on pH adjustment, that is, by changing pH of an amplification system to regulate denaturing—annealing of nucleic acids so as to effectively perform nucleic acid amplification. The inventors completed the present invention based on such discovery.

Terms

As used herein, term “PCR reaction” refers to a polymerase chain reaction.

As used herein, term “primer(s)” refers to a nucleotide which can serve as a starting point of DNA synthesis at a certain condition wherein synthesis of a primer extended product complementary to a nucleic acid chain is induced on a nucleic acid template, that is to say, in the presence of four different nucleoside triphosphates and an agent for polymerization (i.e. DNA polymerase or reverse transcriptase) and in an appropriate buffer and at suitable temperature. The primer is preferably a single-stranded DNA. The appropriate length of primer is typically 15-40 nucleotides. A primer need not reflect the exact sequence of the template and, therefore, a group of similar target molecules can be used as synthesis templates (of consensus amplicon) by changing the combining (reaction) temperature. In order to bind it onto a solid phase or for other purposes, a group having chemical characteristics can be attached to oligonucleotide of primers. The primer sequences can be designed by using a primer sequence design software, such as Primer5.0. In particular, in the present invention, the CG content of primer fragment should not be too high since the CG bases are more difficult to be interrupted.

Term “BR buffer” refers to Britton-Robinson buffer, wherein the buffer is consisted by a mixture of phosphoric acid, boric acid and acetic acid in which a different amount of sodium hydroxide is added. Different amounts of sodium hydroxide may be added to the mixture, thus forming a pair of adjusting buffer of which the pH is 1-14. A preferred Britton-Robinson buffer (H₃PO₄-HAc-H₃BO₃) is prepared by 0.04 mol/L phosphoric acid, boric acid and acetic acid, and is adjusted to a desired pH value by 0.2mol/L NaOH on acidometer.

Nucleic Acid Amplification Test by Acid-Alkaline Method

The present invention provides a method to denature—anneal a nucleic acid by adjusting pH value of the system which comprises:

a method to denature—anneal a nucleic acid which comprises:

adjusting pH of the reaction system to pH 10-14 to denature DNA;

adjusting pH of the reaction system to pH 5-8 to anneal DNA;

wherein the denaturing—annealing process can be conducted repeatedly.

In another preferred embodiment, the denaturing—annealing process can be repeated for at least 2 cycles, preferably at least 10 cycles, more preferably at least 20 cycles, and most preferably at least 30 cycles.

The experiments of the present invention show that acid and alkaline solutions of certain pH value can denature and anneal DNA, and such process is reversible.

Nucleic Acid Amplification by Acid-Alkaline Method

The present invention provides a method to amplify a nucleic acid by adjusting pH value of the reaction system and, more specifically, the method comprises the following steps:

(1a) a denaturation step: in an alkaline condition of pH 10-14, unwinding a double-stranded nucleic acid molecule in an amplification system;

(1b) an annealing and extension step: at a neutral or near-neutral condition of pH 5-8, annealing a single-stranded nucleic acid molecule and the primer in the amplification system and in the presence of a nucleic acid polymerase, extending the primer binding to the single-stranded nucleic acid molecule, thereby forming an amplified double-stranded nucleic acid molecule.

In another preferred embodiment, the method comprises repeating steps (a) and (b) for at least 15-60 times, and preferably 20-45 times.

In another preferred embodiment, in the steps (a) and/or (b), the reaction temperature is 10-70° C. and preferably, the reaction temperature is 15-45° C.

In another preferred embodiment, during the entire amplification process, the temperature of amplification system maintains at 10-70° C. and preferably at 15-45° C.

In another preferred embodiment, the pH of step (a) is preferably 11.5-12.5.

In another preferred embodiment, the pH of step (b) is preferably 6.5-7.5.

In each steps, the preferable time range is as follows: step (a) >20 seconds, and step (b) >60 seconds.

In step (a), it is preferably 20-120s, more preferably 25-80s, and most preferably 35-60s; and in step (b), it is preferably 60-300s, more preferably 60-150s, and most preferably 100-150s. Preferably, it is reacted for a comparatively long time during the first denaturation so as to ensure a complete unwinding.

The nucleic acid can be any double-stranded nucleic acid, which comprises DNA, RNA, or DNA-RNA hybrid molecules.

The amplification system can comprise any other raw materials needed for amplification, such as dNTPs, primers, polymerase, etc. Preferably, the amplification system comprises DNA to be amplified, dNTPs, primers, polymerase and magnesium ion.

During the amplification process, pH value of the amplification system can be tracked so as to detect the progress of amplification system. The pH value of system can be determined by common methods, such as adding indicator or using electrochemical methods. In another preferred embodiment, in step (a) and/or step (b), it further comprises determining electric potential of the amplification system by electrochemical methods so as to determine pH value of the amplification system.

In another preferred embodiment, in step (a) and/or step (b), pH value of the amplification system is adjusted by adding alkaline solution or acid solution. In another preferred embodiment, in step (a) and/or step (b), it further comprises adjusting electric potential of the amplification system by electrochemical methods so as to adjust pH value of the amplification system.

In another preferred embodiment, the method of amplifying nucleic acid is polymerase chain reaction (PCR).

PCR Reaction

Polymerase chain reaction, of which the abbreviation is PCR, is a molecular biological technique to amplify certain DNA fragment.

PCR technology is consisted of three basic reaction steps, denaturation—annealing—extension. Conventional PCR comprises unwinding DNA in vitro in 95 degree, annealing primers and single strand based on paring of complementary bases in 55 degree, and adjusting the temperature to about 72 degrees to synthesize a complementary strand along the phosphate to pentose (5′-3′) orientation by a DNA polymerase. Template DNA is heated to about 93° C. for a certain time, the double-stranded template DNA or the double-stranded DNA formed by PCR amplification are unwound to form a single strand so that it can bind to a primer. After the template DNA is warmed to denature into a single strand, the temperature is lowered to about 55° C. so that the primer and the complementary sequence of single-stranded template DNA are paired and bind together. Under the action of TaqDNA polymerase, the DNA template—primer complex synthesizes a new semi-conservative replication strand which is complementary to template DNA strand, by using dNTP as raw materials and using target sequence as a template and according to complementary pairing of bases and semi-conservative replication principle. More “semi-conservative replication strand” can be obtained by repeating denaturing—annealing—extending cycle.

Denaturing and annealing process for nucleic acid amplification require considerable accuracy and stability of template nucleic acid in every denaturation, annealing and amplification. So it requires that the denaturing and annealing process of nucleic acid is reversible or substantially reversible.

The PCR technology of the present invention uses alkaline and acid solution of certain pH value instead of processes such as raising temperature by heating to affect DNA conformation, thus making DNA molecules denature and annealing.

Since pH changes of the system is more quickly and easier than temperature change, the PCR technology of the present invention can greatly improve the efficiency of PCR reaction. In a preferred embodiment of the present invention, a single PCR cycle needs only 1 to 2 minutes, which reduces nearly 3/4 of time required for a single PCR cycle in conventional technology.

The use of electrochemical working station makes pH of the reaction solution reach the desired range within ten seconds, thus further shortening the time required for a reaction cycle. Further, it is of high reproducibility and has good reaction results.

In another preferred embodiment, the PCR reaction of the present invention comprises the following steps:

(1a) a denaturation step: in an alkaline condition of pH 10-14, unwinding a double-stranded nucleic acid molecule in an amplification system;

(1b) an annealing and extension step: at a neutral or near-neutral condition of pH 5-8, annealing a single-stranded nucleic acid molecule and a primer in the amplification system, and in the presence of a nucleic acid polymerase, extending the primer binding to the single-stranded nucleic acid molecule, thereby forming an amplified double-stranded nucleic acid molecule.

In the present invention, DNA fragments of different sizes were amplified based on the property of the electrochemical working station. The results were entirely consistent to those of conventional PCR amplification. However, the reaction time of the electrochemistry PCR reaction in the present invention was only 40 minutes, which was reduced about 3/4 when compared with the conventional PCR.

In the present invention, there is no limitation to the appropriate polymerases, and it can be any commercially available nucleic polymerase, or any nucleic polymerase prepared by conventional methods. Representative examples include (but are not limited to): E. coli DNA polymerase I, Klenow fragment (DNA polymerase I large fragment), E. coil DNA polymerase II, E. coli DNA polymerase III, T4 DNA polymerase, T7 DNA polymerase, DNA polymerase a, DNA polymerase f3, DNA polymerase γ, DNA polymerase β, Taq DNA polymerase, Tth DNA polymerase, pfu DNA polymerase, Vent DNA polymerase, Bca Best DNA polymerase, Sac DNA polymerase, Iproof DNA polymerase, KOD DNA polymerase, Phusion DNA polymerase, U11traPF™ DNA polymerase, LA Tag DNA polymerase, and Super Tag DNA polymerase.

In the present invention, a particularly preferable polymerase is a polymerase which can tolerate or essentially tolerate pH value fluctuations during denaturing and annealing. Of course, if pH tolerance of polymerase is comparatively poor, a more moderate pH condition can be adopted, or supplementary polymerase can be added during amplification.

Preferably, 1-10U (international standard unit) of enzyme is used in each PCR reaction cycle.

Generally, the quantity of enzyme added may be greatly excessive and depends on reaction volume. Preferably, the volume of the enzyme liquid should be less than 10% of reaction volume.

In another preferred embodiment, during the cycles, enzyme in small volume and high concentration is added to ensure that the scale of the reaction system is basically constant.

Electrochemical Working station (A Method to adjust pH with Potential)

The present invention is based on the combination of an electrochemical working station and a micro-fluidic chip, wherein a pH change is achieved by changing potential difference between a working electrode and a reference electrode on the chip to produce an electric current which causes a corresponding degree of decomposition of electrolytes in DNA reaction solution. Specifically, since there are a large number of electrolytes in DNA solution, they release acidic and basic ions through hydrolysis effect under an applied electric field. The micro-fluidic chip comprises Ag/AgCl electrode (working electrode) and IrO₂ electrode (reference electrode), and a reaction area containing DNA reaction solution (R area) and /or a control area containing electrolyte solution (C area).The working electrode connects to the C area, while the reference electrode connects to the R area. The C area and the R area are semi-closed connected by 1% agarose, that is to say, the current can get through the agarose, while the solutions in these two regions are mutually not connected.

When an electrochemical working station and a micro fluidic chip are connected by wire, the potential difference between the working electrode and the reference electrode in the condition of required pH value is first determined by an open-circuit potential method. After that, the potential difference is set in the electrochemical working station to produce the corresponding feedback current. Once the potential difference between the working electrode and the reference electrodes of the micro fluidic chip is formed, the corresponding current is generated so that the electrochemical working station produces a relatively big feedback current, thus causing water electrolysis reaction at the working electrode and producing electrolytic ions in C zone. Accordingly, based on the principle of charge balance, in R zone, the pH value of the solution in which DNA exists in changes oppositely, thus causing DNA conformational changes.

A sketch of electrochemical—micro fluidic chip device is shown in FIG. 5.

Nucleic Amplification Equipment

The nucleic amplification equipment of the present invention comprises:

(3i) an device to place a vessel in which a nucleic acid amplification reaction is conducted, wherein the vessel is used to accommodate an amplification system for conducting a nucleic acid amplification reaction;

(3ii) an alkaline solution adding device which is used to add alkaline solution into the amplification system so as to adjust pH of the amplification system to alkaline condition;

(3iii) an acid solution adding device which is used to add acid solution into the amplification system so as to adjust pH of the amplification system to acidic condition;

(3iv) a pH determining device which is used for accurately controlling pH value of the system.

In another preferred embodiment, one or more devices in (i)-(iv) are included in an electrochemical working station.

The device to place the vessel in which the nucleic acid amplification reaction is conducted can be any suitable device, such as a culture flask, a centrifuge tube, etc. Preferably, the device is a DNA reaction chip.

The pH determining device can be any device available in the art, including an indicator, an acidometer, or a pH potentiometer. Preferably, the device determines the pH value of the system by an electrochemistry method.

The equipment can comprise any other necessary devices, such as a polymerase adding device so as to add a polymerase into the system during the amplification procedure.

More preferably, the equipment is a system of electrochemical working station-DNA reaction controlling chip, which comprises:

(4i) an amplification device to place a vessel in which a nucleic acid amplification reaction is conducted, wherein the vessel is used to accommodate an amplification system for conducting a nucleic acid amplification reaction; and

(4ii) an electrochemical working station which is used to regulate and detect pH of the reaction system.

In another preferred embodiment, the amplification device comprises a micro fluidic chip, a DNA reaction chip, an eppendorf tube, or a culture flask.

In another preferred embodiment, the electrochemical working station connects to the device by wire.

In another preferred embodiment, the electrochemical working station adjusts pH of the amplification system to a desired value by applying a predetermined voltage to the system.

In a preferred embodiment of the present invention, the DNA amplification of DNA fragment using the above electrochemical working station-DNA reaction controlling chip system comprises the following work steps:

(1) connecting Ag/AgCl wire of a DNA reaction chip to a working electrode of an electrochemical working station, wherein an Ir0₂ wire is connected to a reference electrode and an Ir wire is connected to a counter electrode;

(2) formulating a series of Britton-Robinson (BR) buffer solution of which the pH values are from 1 to 13, and successively adding them dropwise into a reaction zone of chip so as to gradually change pH of the reaction zone;

(3) determining a potential difference between IrO₂ electrode and Ag/AgCl electrode by an open circuit potential electrochemical method, thus determining a correlation curve of the potential difference and the pH value of the solution;

(4) adding a DNA template to be amplified into the DNA chip reaction zone, and then adding primers, dNTPs, magnesium ion, and DNA polymerase I (Using DNA polymerase I as an amplification enzyme is based on that the whole electrochemical amplification reaction is conducted at room temperature, because the cost of using DNA polymerase I at normal temperature conditions is lower than that of Taq enzyme);

(5) applying the corresponding particular potential to the working electrode based on data of the correlation curve between pH and IrO₂ electrode, so as to achieve rapid regulation and stabilization of specific pH value, thereby controlling the pH sensitive DNA amplification by changing the pH value between pH 7 and pH 12 constantly;

(6) controlling the electrochemical working station so as to make pH of the reaction zone reaches pH 12 and maintaining for 45 seconds, then returning to pH=7 and maintaining for 45 seconds, and at the same time, adding new DNA polymerase I.

This is one cycle, and totally there are 30 cycles.

(7) The final products are detected by gel electrophoresis together with conventional PCR products.

(8) In addition to electrophoresis detection, the product amplified by electrochemical working station-DNA reaction controlling chip can be sequenced, or detected by digestion with a restriction enzyme targeting at a specific site.

It is shown by electrophoresis detection that electrochemical working station-DNA reaction controlling chip can fast, efficiently and accurately amplify DNA.

Nucleic Acid Detection Methods

The present invention also provides a method to detect a nucleic acid, wherein the method comprises:

Amplifying a nucleic acid to be detected by the method of the present invention and detecting the amplified nucleic acids.

The amplified nucleic acid can be detected by any conventional method in the art, such as detected by an electrophoresis method. In another preferred embodiment, before detection, the products are digested with restriction enzyme, and then are used for subsequent detection.

The main advantages of the present invention include:

(a) The method of the present invention can conduct nucleic amplification under room temperature without heating—cooling cycle so that it is of less energy consumption and energy saving.

(b) Since the temperature change is a slow procedure, the total PCR reaction time of traditional PCR technology is at least 2 hours. The use of electrochemical working station can make pH of the reaction solution accurately reach the desired range within ten seconds of time, thus greatly improving efficiency. The reaction time of electrochemical PCR is just 40 minutes, which is reduced by nearly three-fourth when compared with conventional PCR. Therefore, acid-base DNA amplification technology based on the electrochemical working station-DNA reaction controlling chip is another breakthrough PCR reaction technology innovation.

(c) In the present invention, DNA fragments of different sizes were amplified. The results were entirely consistent to those of conventional PCR amplification. The method of invention has consistent repeatability and good reaction results.

The present invention will be further illustrated below with reference to the specific examples. It should be understood that these examples are only to illustrate the invention but not to limit the scope of the invention. The experimental methods with no specific conditions described in the following examples are generally performed under the conventional conditions, or according to the manufacture's instructions. Unless indicated otherwise, parts and percentage are calculated by weight.

### EXAMPLE 1

### DNA Denaturing—Annealing Circulation Test

1.1 Designed Method

This example is to determine reversibility of the denaturing and annealing of nucleic acid during the acid-base amplification method. Specifically, in the present example, random DNA sequences were denatured in an alkaline solution of pH=14 for different periods to dissociate the double-stranded DNA; then were annealed in a neutral solution of pH=7 for different periods to reform a double strand.

DNA samples from different stages were detected by electrophoresis and analyzed.

As a control, DNA fragments only treated with pH=14 basic solution to denature but not treated with pH=0 acidic solution to anneal showed no band after electrophoresis, which indicated that DNA samples treated with strong alkali could not form a visible band in the gel because the double bonds in the sample solution are in dissociated state.

If the denaturalized and renaturalized DNA fragments were at the same position as that of the same DNA fragment without any treatment in electrophoresis, then annealed DNA after being treated with strong alkali was not destroyed, and the acid-base treatment could make DNA reversibly denature and anneal.

If DNA after denaturing and annealing (including twice denatured DNA) and the non-denatured DNA as a positive control were at the same position in electrophoresis and the bands had essentially consistent clarity; meanwhile, DNA without being annealed produced no band, it showed that the DNA was not destroyed after being treated with strong acid and strong alkali. However, DNA samples only treated with alkali could not form a visible strip since their double bonds are in disconnected state. So it further proved that the DNA could be reversibly denatured and annealed by acid and base.

1.2 Experimental Procedure

5 μg of soybean Lectin DNA was dissolved by 35 μl pH 14 NaOH solution. The solution was divided into 7 aliquots and each is 5 μl. The seven aliquots of solution were treated as follows:

(1) After three minutes, into the solution was added with 5 μl of HCl (pH=0) to treat for 90 seconds, and then added with 100% ice ethanol to precipitate the DNA.

(2) After 3 minutes, 100% ice alcohol was added to participate the DNA.

(3) After two minutes, into the solution was added with 5 μl of HCl (pH=0) to treat for 90 seconds, and then added with 100% ice ethanol to precipitate the DNA.

(4) After two minutes, into the solution was added with 5 μl of HCl (pH=0) to treat for 90 seconds, and then added with 5 μl of NaOH solution (pH=14), then added with 100% ice ethanol to precipitate the DNA.

(5) After one minute, into the solution was added with 5 μl of HCl (pH=0) to treat for 90 seconds, and then added with 100% ice ethanol to precipitate the DNA.

(6) After 1 minute, 100% ice alcohol was added to participate the DNA.

(7) After 30 seconds, into the solution was added with 5 μl of HCl (pH=0) to treat for 90 seconds, and then added with 100% ice ethanol to precipitate the DNA.

After the treatment was finished, each sample was detected with 1% agarose gel electrophoresis. The detection results were shown in FIG. 1, wherein the strips 1-7 were successively the above samples Nos. 1-7, and the strip No. 8 was the positive control, i.e., DNA fragment untreated.

TABLE 1
Denaturing-annealing time table in Example 1

| | Time of denaturing | Time of annealing after |
|---|---|---|
| Number of | after adding NaOH | adding HCl solution |
| lane | solution (pH 14) (sec) | (pH 0) (sec) |
| 1 | 180 | 90 |
| 2 | 180 | 0 |
| 3 | 120 | 90 |
| 4 | 120 + 120 | 90 |
| | (denaturalized twice) | |
| 5 | 60 | 90 |
| 6 | 60 | 0 |
| 7 | 30 | 90 |
| 8 | 0 | 0 |

1.3 Results

The results were shown in FIG. 1, which were completely consistent with our expectation. DNA after denaturing and annealing (including twice denatured DNA) and the non-denatured DNA as a positive control were at the same position in electrophoresis and the bands had essentially consistent clarity. Meanwhile, DNA without being annealed produced no band. It suggested that the DNA was not destroyed after being treated with strong acid and alkali, while DNA samples only treated with alkali could not form a visible strip since their double bonds were in disconnected state. Therefore, the DNA could be reversibly denatured and annealed by acid and alkali.

More experiments have showed that, when the denaturation time is over 3 minutes (e.g., 5 minutes, 10 minutes, 15 minutes), the 90 seconds' annealing period can also get an good effects. In other words, the DNA can be well denatured and annealed without any obvious damages. Correspondingly, for different denaturation periods, the extension of annealing periods (e.g., 5 minutes, 10 minutes, 15 minutes) can also achieve an ideal effect.

### EXAMPLE 2

### Appropriate pH Value range of DNA Denaturation in Acid-Base Method

This example was to determine the pH value range of the denaturing and annealing of a nucleic acid during the acid-base amplification method. Specifically, in this example, a particular DNA sequence was denatured and annealed in different pH, and amplified. Then the product was detected by electrophoresis.

The design of this experiment was as follows. DNA was denatured under different alkaline pH conditions and acidic solution was added to anneal DNA under neutral condition of pH 7, thus determining the alkaline pH range for DNA renaturation. The buffer used in the example was BR buffer. The pH of the denaturing-annealing buffer pair comprised: denaturing solution pH 14, annealing solution pH 0; denaturing solution pH13, annealing solution pH 1; denaturing solution pH12, annealing solution pH 2; denaturing solution pH11, annealing solution pH 3; denaturing solution pH 10, annealing solution pH 4, totally five different combinations.

The fragment to be amplified in this example was soybean Lectin gene (608 bp). The specific steps were as follows:

(1) Add 20 ng of Lectin gene and the corresponding primers into a tip tube, and then add 1.5mM of Mg²⁺, 200 μM of dNTPs. The starting volume was kept as 20 μl, the solvent was Q water, of which the pH was 7.

(2) 15 μl of BR buffer (pH=14) was added into the mixture, and was stood for 180 seconds.

(3) 15 μl of BR buffer (pH=0) was added, the solution was adjusted to neutral and stood for 90 seconds. Meanwhile, 0.2 μl of Taq enzyme (0.1U) was added.

(4) The steps of (1)-(3) were repeated, for every 2 times, 100% of ice alcohol was added to participate DNA. The precipitate was separated out and resolved, and then the steps (1)-(3) were repeated.

(5) The above steps were repeated for 30 times.

(6) Treat the soybean Lectin gene sequence with BR buffers pairs comprising denaturing solution pH13, annealing solution pHl; denaturing solution pH 12, annealing solution pH 2; denaturing solution pH 11, annealing solution pH 3; and denaturing solution pH 10, annealing solution pH 4. The steps were the same as steps (1)-(5).

(7) The product obtained was detected by 1% agarose gel electrophoresis.

As shown in FIG. 2, the basic denature effect were observed in BR buffers of which the pH value was 14, 13, 12, 11, or 10. After 180 seconds, annealing was conducted with BR buffers of which the pH value was 0, 1, 2, 3 and 4. The annealing time and the amplification time were both 90 seconds. (The standard were set in accordance to the maximum annealing time in the experiment). For each pH buffer pair (pH14/0, 13/1, 12/2, 11/3, 10/4), the fragment was amplified according to the above method repeatedly for 30 times, and was detected by 1% agarose gel electrophoresis. Numbers 1-4 respectively represent the amplification effect of DNA template using BR buffer solutions of which the pH was 10/4, 11/3, 12/2, 13/1, 14/0.

Under the above five pH denaturation/annealing conditions (denaturing solution pH 13, annealing solution pH 1; denaturing solution pH 12, annealing solution pH 2; denaturing solution pH 11, annealing solution pH 3; and denaturing solution pH 10, annealing solution pH 4), there were obvious DNA amplification strips obtained in the DNA amplification experiment, suggesting that the method could be conducted in those denaturing/annealing pH values, i.e., the pH range of DNA denaturing in the acid-base amplification method was pH>10.

According to the result, all of the pH=10-14 BR buffers could unwind DNA double stranded structure, wherein when pH=12, the unwinding effect of DNA double stranded structure was most significant and the condition was comparatively gentle. Therefore, the most suitable pH value for denaturing and annealing of the double stranded nucleic acid was 12/2 (i.e., using the BR buffer of pH 12 as a denaturing solution, and the BR buffer of pH 2 as an annealing solution. Hereinafter, the similar expressions are used).

TABLE 2
Description of the legends of electrophoresis

| Number of | pH value of | pH value of |
|---|---|---|
| lane | denaturing BR buffer | annealing BR buffer |
| 1 | 14 | 0 |
| 2 | 13 | 1 |
| 3 | 12 | 2 |
| 4 | 11 | 3 |
| 5 | 10 | 4 |

### EXAMPLE 3

### Appropriate pH Value Range of DNA Annealing in Acid-Base Method

It was proved in the example 2 that the acid-base amplification method can be conducted at a denaturation pH>10.This example further determined the preferred annealing pH value range for amplifying DNA by the acid-base method. Specifically, after denaturing DNA sequence in several different pH (pH>10) BR buffer pairs, the DNA was annealed in different pH values (pH=5-8) to test the annealing effect of DNA in pH range of 5-8.

The soybean Lectin DNA was denatured by a pH 14 BR buffer solution. Then the pH value was adjusted so that the pH of soybean Lectin DNA annealing solution were respectively 5, 6, 6.5, 7, 7.5 and 8. After that, the treated solutions were detected by 1% agarose gel electrophoresis to determine the appropriate annealing pH value range of acid-base amplification method. The results were shown in FIG. 3, wherein numbers 1-6 respectively represented the annealing effects of the soybean Lectin DNA when the pH values of the annealing solutions were 5, 6, 8, 6.5, 7, and 7.5. The result showed that when the pH value was 5-8, the annealing of the soybean Lectin DNA could be conducted, wherein the annealing effect was better when the pH value of the annealing is 6.5-7.5.

The pH 5-8 annealing solution could anneal DNA as well when a buffer solution or a sodium hydroxide solution of which the pH was 13, 12, 11 or 10 were used to denature DNA. The annealing effect was better when the pH value of the annealing solution was 6.5-7.5.

TABLE 3
Description of legends of electrophoresis

| | pH value of | pH value of |
|---|---|---|
| No. of lane | denaturing solution | annealing solution |
| 1 | 14 | 5 |
| 2 | 14 | 6 |
| 3 | 14 | 8 |
| 4 | 14 | 6.5 |
| 5 | 14 | 7 |
| 6 | 14 | 7.5 |

### EXAMPLE 4

### Appropriate Time Range of DNA Denaturing, Annealing and Amplification in Acid-Base Method

The BR buffer (pH=12/2) was used in the detection of amplification time of soybean Lectin gene so as to determine the appropriate range of the DNA denaturing time and annealing time in the acid-base amplification method. The detection process was the same as that of Example 2, wherein the Variables to be controlled were the denaturing and annealing time. On the basis of Example 1 and a series of pre-examples, in this example, the denaturation starting time was set at 25 seconds while one gradient was set at 5 seconds, and the terminal time was set at 55 seconds. The annealing time was set at 65 seconds while one gradient was set at 20 seconds, and the terminal time was set at 125 seconds. At the end of annealing, 100% ice ethanol was added to precipitate DNA.

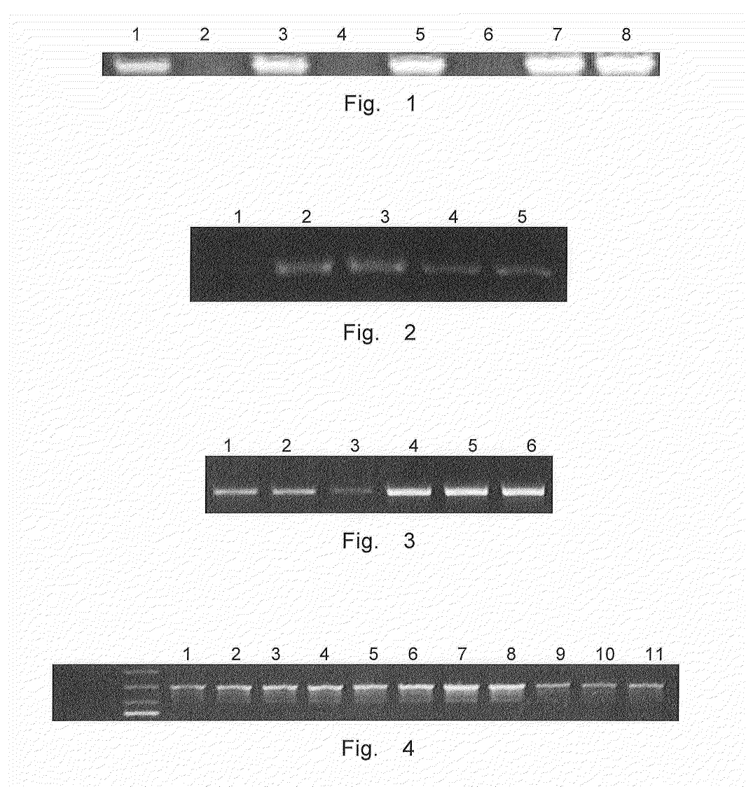

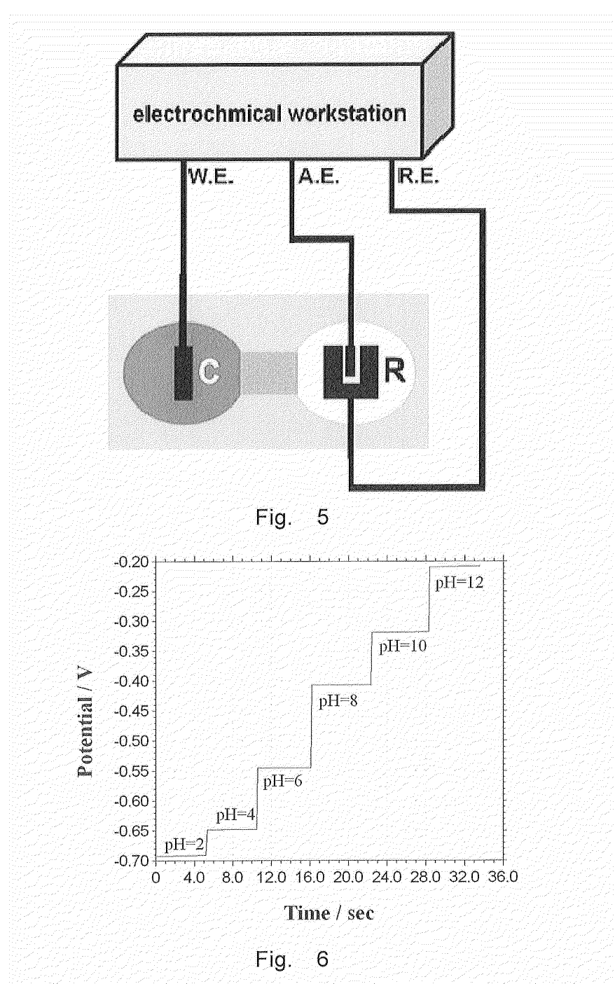

FIG. 4 shows the result of a typical experimental group, wherein in the experimental group, the denaturing and annealing pH value were set by referring to the best results shown in Example 2, i.e., the denaturing pH was 12, and the DNA annealing was conducted by adjusting pH to 7 with a pH 2 acidic solution. Lanes 1-11 were under different reaction conditions. The detailed parameters were shown in the following table:

TABLE 4
Description of legends of electrophoresis

| | | Time of annealing and |
|---|---|---|
| No. of lane | Time of denaturing | amplifying |
| 1 | 30 | 65 |
| 2 | 30 | 85 |
| 3 | 30 | 105 |
| 4 | 30 | 125 |
| 5 | 25 | 125 |
| 6 | 30 | 125 |
| 7 | 35 | 125 |
| 8 | 40 | 125 |
| 9 | 45 | 125 |
| 10 | 50 | 125 |
| 11 | 55 | 125 |

The results showed that the DNA amplification could all be completed when the time of denaturation was 25-55 seconds and the time of annealing was 65-125 seconds. The inventor also tried to denature for 2 min and to anneal and amplify for 5 min, and the DNA amplification could also be completed. Denaturing for 35 seconds and annealing and amplifying for 125 seconds was a denaturing—annealing combination having best effect as well as shortest time period.

The inventor also tried DNA amplification experiments under denaturing pH range of 10-14 (under pH=10, 11, 13 or 14) and annealing pH range of 6-8 (under pH=6, 6.5, 7, 7.5 or 8). The above denaturing—annealing and amplifying time period results were also applicable.

### EXAMPLE 5

### Preparing an Electrochemical Micro Fluidic Chip for pH-PCR Reaction

The sketch of the equipment is shown in FIG. 5. The electrochemical working station was connected to a micro-fluidic chip by wire. The micro-fluidic chip comprised Ag/AgCl electrode (working electrode), Ir electrode (counter electrode) and Ir0₂ electrode (reference electrode), and a reaction area containing DNA reaction solution (R area) and /or a control area containing electrolyte solution (C area).The working electrode connected to the C area, while the reference electrode connected to the R area. The C area and the R area were semi-closed connected by 1% agarose (i.e., the current could get through the agarose, while the solutions of the two regions were not connected). The counter electrode, the working electrode and the electrochemical working station together formed a current loop, which would not influence the status of the electric current. The potential difference between the reference and working electrodes was formed due to flow of charged ions, and then a corresponding ion gradient in the R zone was formed, thereby resulting electrolysis of the aqueous solution in R area and the pH value change of solution. The purpose of this system was to control pH value of the R zone by electrochemical reaction, thereby controlling the DNA denaturing and annealing therein.

### EXAMPLE 6

### Establishing a pH-PCR Method Based on the Electrochemical Method

The specific implementation process was in accordance to the method of the invention. The soybean Lectin DNA plasmid (TAKARA Co.) was bought. Primers were designed by the primer design software primer5.0. A sequence of 541 bp on the plasmid was amplified by conventional PCR method and used as a template. The electrochemical chip was balanced by lmol/L KCl solution on the day before experiment, and hydrochloric acid was used to solve the oxide on the surface. The working electrode, reference electrode and the counter electrode were connected correctly. Agarose was used to enclose an area between the reaction area and control area. The reaction area was added with 5 μl of temperate, 6 μl of each primer, 3 μl of Mg²⁺, 5 μl of dNTP , and 30 μl of pure water, while 0.2 μl DNA polymerase I was added in each cycle. A KCl solution was added into the control area and a circuit passage was formed.

Firstly, the relationship between open circuit potential of the control area and the reaction area and the pH value was determined. That is to say, BR buffers of different pH value were added into the reaction area before adding the reaction solution drop wise. The standard curve was determined with “Open Circle Potential”. The pH value determined was from 2 to 12.

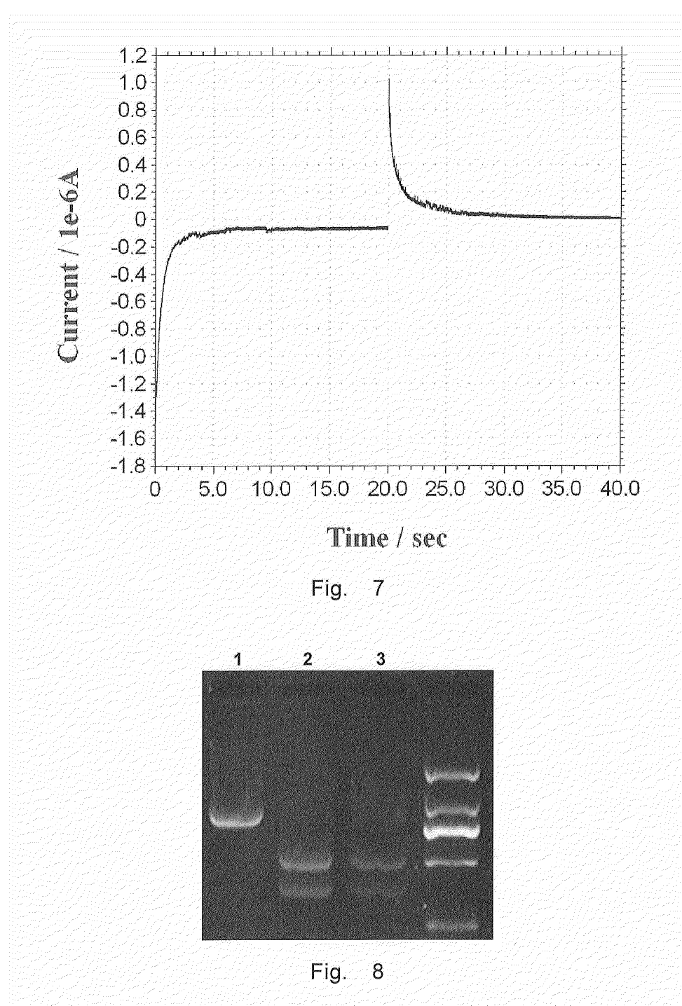

FIG. 6 shows the relationship between the control voltage of electrochemical working station -DNA reaction chip and the pH value of the solution. The correspondence relationship was of good linear relation with R²=0.9996.

FIG. 7 shows the relationship between the current variation and the solution pH value during one cycle of amplifying DNA template by an electrochemical method. During the first 20 seconds, the negative current decreased gradually and approached zero, while the solution was alkaline; during the latter 20 seconds, the positive current decreased and approached zero, while the solution was acidic.

After the standard curve was determined, the voltages corresponding to pH=12 and pH=7 were set based on the corresponding voltage of different pH values, and the electrochemical denaturing-annealing was conducted according to “i-t curve”. The working voltages used were 200-180mv (pH=12), and −500 to −450 mv (pH=7). The DNA denaturation time for pH=12 was 45 seconds, and DNA annealing and extending time for pH=7 was 105 seconds. The reaction comprised 30 cycles.

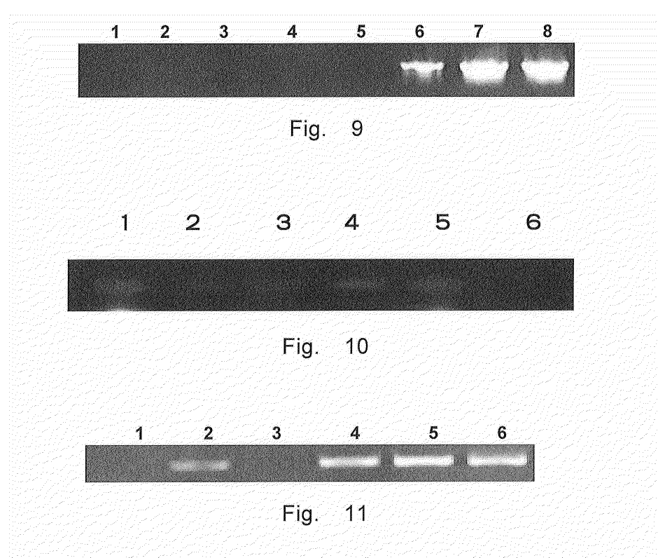

PCR products were detected with 1% agarose gel electrophoresis, and the gel electrophoresis of the amplified products was shown in FIG. 9, wherein numbers 1-8 respectively represents DNA template, electrochemical amplification in 37° C. environment without DNA polymerase I, DNA amplification without dNTPs, electrochemical amplification without primers, electrochemical amplification added DNA polymerase I at 9° C. environment, electrochemical working station -DNA reaction chip amplification, conventional PCR amplification, conventional PCR amplification in which the product amplified by the electrochemical working station -DNA reaction chip was used as a template.

TABLE 5
Description of the legends of electrophoresis

| No. of lane | The Illustration of Electrophoresis |
|---|---|
| 1 | DNA template, no amplification |
| 2 | Electrochemical amplification in 37° C. environment |
| | without DNA polymerase I |
| 3 | DNA amplification without dNTPs |
| 4 | Electrochemical amplification based on DNA |
| | polymerase I, without primers |
| 5 | Electrochemical amplification at 9° C. based on Taq |
| | enzyme |
| 6 | Electrochemical amplification at 37° C. based on |
| | DNA polymerase I |
| 7 | Conventional PCR amplification |
| 8 | Conventional PCR amplification in which the |
| | product amplified by electrochemical working |
| | station -DNA reaction chip was used as a template |

In similar experiments, electrochemical pH-PCR reactions with various parameters were determined. The results showed that, similar to the manual operation

PCR, a better DNA amplification result was obtained when the condition was as follows: denaturation pH>10, annealing pH<8, denaturing time>25 seconds, and annealing time>65 seconds.

### EXAMPLE 7

### Detection and Identification of Electrochemical-Based pH-PCR Amplification Product

In Example 6, after the reaction was completed, the reaction solution in the reaction zone was collected. DNA was precipitated by ethanol and dissolved in pure water. The pH-PCR amplification product and the conventional PCR amplification products were subjected to electrophoresis detection, digestion detection and sequencing detection.

There was a Hinf I enzyme cleavage site on the 388 bp from 5′ end of Soybean Lectin gene which could be identified by the restriction enzyme. Digestion comparison of electrochemical amplification product and conventional PCR amplification product was conducted by using Hinf I restriction enzyme, and the detection of the digested product was conducted by 1% agarose gel electrophoresis. The electrophoresis detection results are shown in FIG. 8. Number 1-3 respectively represent soybean Lectin DNA sequence, the result of digested traditional PCR product, and the result of digested electrochemical amplification product. The results showed that the digested fragments of the electrochemical amplified DNA are of the same size as that of the traditional PCR, suggesting that nucleotide sequence of the amplification product based on electrochemical pH-PCR conformed to the nucleotide sequence of the DNA template.

The resultant fragments of the electrochemical amplification were sequenced. The results further confirmed that nucleotide sequence of the amplification product based on electrochemical pH-PCR conformed to the nucleotide sequence of the DNA template, and the method could accurately amplify a template DNA.

### EXAMPLE 8

### Amplification of Soybean Lectin Gene with Different Types of DNA Polymerase in an Electrochemical Working Station -DNA Reaction Control Chip

The specific experimental method was the same as that in Example 6, except that during the annealing period of each cycle, the following enzymes was added as a DNA duplicate polymerase: E. coil DNA polymerase I, E. coli DNA polymerase II, E. coli DNA polymerase III, Bca Best DNA polymerase and UlltraPF™ DNA polymerase. The polymerase used was selected randomly. The amplification reaction was conducted in the electrochemical working station-DNA reaction control chip for 30 cycles. All the cycle amplification products were detected by 1% agarose gel electrophoresis. FIG. 10 shows the detection results of agarose electrophoresis. Numbers 1-5 respectively represent the results for E. coli DNA polymerase I, E. coli DNA polymerase II, E. coli DNA polymerase III, Bca Best DNA polymerase, and UlltraPF™ DNA polymerase when they were used as polymerase for electrochemical working station -DNA reaction control chip amplification. Number 6 represented the result when no polymerase was added during the whole amplification procedure. The results showed that all DNA polymerases commonly used in the conventional PCR were suitable for the present DNA amplification based on change of pH value.

### EXAMPLE 9

### Electrochemical Working Station -DNA Reaction Control Chip Amplification of Human Genome Chromosome 6th Gene Fragment

The whole length of the genome fragment was 1187 bp, while the encoding gene was related to occurrence of human psoriasis. Therefore, it was important to amplify it by new method. Firstly, primers were designed according to the sequence. The primers all had a length of 25 bp, and the software used for designation was Primer 5.0. The primers were synthesized by TAKARA Company. The same method in Example 2 was used to treat the electrochemical working station and DNA reaction control chip. In the specific experiment, the DNA denaturing and annealing times were set at 1 minute and 2 minutes in accordance to the above. Since the fragment to be amplified was relatively long, the denaturing and annealing time were increased. (The conventional PCR needs 6 hours for amplification).The pH values corresponding to the voltage were still pHl2 and pH2. After the reaction was completed, the DNA was participated with alcohol, and solved in pure water. This amplified product and the conventional PCR product were detected by electrophoresis.

The results are shown in FIG. 11. Numbers 1-6 respectively correspond to DNA template, manual operated amplification based on BR buffer, electrochemical DNA amplification without dNTPs, electrochemical amplification in 37° C. environment, conventional thermotropy PCR amplification, conventional PCR amplification in which the product amplified by the electrochemical working station -DNA reaction chip was used as a template.

TABLE 6
Description of the legends of electrophoresis

| No. of lane | Illustration of Electrophoresis |
|---|---|
| 1 | DNA template |
| 2 | manual operated amplification based on BR buffer wherein |
| | the amplification enzyme was DNA polymerase I |
| 3 | electrochemical DNA amplification without dNTPs wherein |
| | the amplification enzyme was DNA polymerase I |
| 4 | electrochemical amplification in 37° C. environment |
| | wherein the amplification enzyme was DNA polymerase I |
| 5 | conventional PCR amplification |
| 6 | conventional PCR amplification in which the product |
| | amplified by the electrochemical working station -DNA |
| | reaction chip was used as a template |

The results showed that the DNA amplification method based on electrochemical working station and micro-fluidic and induced by solution pH change could achieve a rapid amplification of template DNA under room temperature. The time of a single cycle was about 150 seconds, while the whole amplification time of 30 cycles was within one and a quarter hour. The amplification product was accurate, thus satisfying the demand for detection of biomolecule. The method is a new DNA amplification method, and the equipment cost, time cost and consumables cost are all lower than those of current PCR technology.

All literatures mentioned in the present application are incorporated herein by reference, as though each one is individually incorporated by reference. Additionally, it should be understood that after reading the above teachings, those skilled in the art can make various changes and modifications to the present invention. These equivalents also fall within the scope defined by the appended claims.

1. A method for amplifying nucleic acid which comprises:
(1a) a denaturing step: in an alkaline condition of pH 10-14, unwinding a double-stranded nucleic acid molecule in an amplification system; and
(1b) an annealing and extension step: at a neutral or near-neutral condition of pH 5-8, annealing single-stranded nucleic acid molecules and primers in the amplification system and in presence of a nucleic acid polymerase, extending the primers binding to the single-stranded nucleic acid molecules, thereby forming amplified double-stranded nucleic acid molecules.

2. The method of claim 1,wherein the nucleic acid polymerase is an enzyme having DNA polymerase activity and, preferably, the nucleic acid polymerase comprises E. coil DNA polymerase I, Klenow fragment (DNA polymerase I large fragment), E. coil DNA polymerase II, E. coil DNA polymerase III, T4 DNA polymerase, T7 DNA polymerase, DNA polymerase a, DNA polymerase (3, DNA polymerase y, DNA polymerase δ, Taq DNA polymerase, Tth DNA polymerase, pfu DNA polymerase, Vent DNA polymerase, Bca Best DNA polymerase, Sac DNA polymerase, Iproof DNA polymerase, KOD DNA polymerase, Phusion DNA polymerase, UlltraPF™ DNA polymerase, LA Tag DNA polymerase, or Super Tag DNA polymerase.

3. An equipment for amplifying a nucleic acid which comprises:
(3i) an device to place a vessel in which a nucleic acid amplification reaction is conducted, wherein the vessel is used to accommodate an amplification system for conducting a nucleic acid amplification reaction;
(3ii) an alkaline solution adding device which is used to add alkaline solution into the amplification system so as to adjust pH of the amplification system to alkaline condition;
(3iii) an acid solution adding device which is used to add acid solution into the amplification system so as to adjust pH of the amplification system to acidic or neutral condition; and
(3iv) a pH determining device which is used for accurately controlling pH value of the system.

4. An equipment for amplifying a nucleic acid, which comprises:
(4i) an amplification device to place a vessel in which a nucleic acid amplification reaction is conducted, wherein the vessel is used to accommodate an amplification system for conducting a nucleic acid amplification reaction; and
(4ii) an electrochemical working station which is used to regulate and detect pH of the reaction system.

5. The equipment of claim 4, wherein the amplifying device is a micro fluidic chip.

6. A method for amplifying a nucleic acid which comprises:
(1) providing an amplification system which comprises a DNA template to be amplified, primers, dNTPs, magnesium ions, and a polymerase; and
(2) adjusting pH value of the amplification system by an electrochemical method so as to achieve denaturing and annealing of nucleic acid molecules, and to conduct amplification of the nucleic acid.

7. The method for amplifying a nucleic acid of claim 6, wherein the method comprises:
in step (1), providing an nucleic acid amplification equipment of claim 4, and adding the DNA template, the primers, dNTPs, magnesium ions, and the polymerase into the equipment so as to form an amplification system; and
in step (2), the electrochemical method is by using an electrochemical working station.

8. A method to amplify a nucleic acid by using an electrochemical method, wherein the method comprises one or more of the following steps:
determining the relationship between electrical parameters and pH of solution in the amplification solution system;
setting the electrical parameters; unwinding double-stranded nucleic acid in the amplification system by controlling pH to an alkaline condition of pH 10-14 through solution electrochemical reaction;
setting the electrical parameters, annealing a single-stranded nucleic acid molecule and a primer in the amplification system, and in the presence of a nucleic acid polymerase, extending the primer binding to the single-stranded nucleic acid molecule to form an amplified double-stranded nucleic acid molecule by controlling pH at a neutral or near-neutral condition of pH 5-8 through solution electrochemical reaction.

9. A method for detecting a nucleic acid which comprises:
amplifying a nucleic acid to be detected by using a method of claim 1; and
detecting the amplified nucleic acid.

10. A method for denaturing and annealing a nucleic acid, wherein the method comprises:
(10a) providing a reaction system containing a nucleic acid;
(10b) adjusting pH of the reaction system to pH=10-14 so as to denature DNA, and adjusting pH of the reaction system to pH=5-8 so as to anneal the DNA;
wherein the denaturing—annealing process can be conducted repeatedly.

11. The method of claim 1, wherein in the steps (a) and/or (b), reaction temperature is 10-70° C.

12. The method of claim 1, wherein the pH in step (a) is 11.5-12.5; and the pH in step (b) is 6.5-7.5.

13. The method of claim 1, wherein in step (a) it is 20-120s; and in step (b) it is 60-300s.

14. The equipment of claim 4, wherein the amplification device comprises a micro fluidic chip, a DNA reaction chip, an eppendorf tube, or a culture flask.

15. The equipment of claim 4, wherein the amplification device is a micro fluidic chip.

16. The equipment of claim 15, wherein the micro fluidic chip comprises a working electrode and/or a reference electrode.

17. The equipment of claim 16, wherein the working electrode is Ag/AgCl electrode.

18. The equipment of claim 16, wherein the reference electrode is 1r0₂ electrode.

19. The equipment of claim 16, wherein the working electrode connects to the C area; the reference electrode connects to the R area; and the C area and the R area are semi-closed connected.

20. A method for detecting a nucleic acid which comprises:
amplifying a nucleic acid to be detected by using a method of claim 6; and
detecting the amplified nucleic acid.

21. A method for detecting a nucleic acid which comprises:
amplifying a nucleic acid to be detected by using a method of claim 8; and
detecting the amplified nucleic acid.
